(19) United States
(12) Patent Application Publication
(10) Pub. No.: US 2002/0110873 A1
(43) Pub. Date: Aug. 15, 2002

(54) Process for the production of lipstatin and tetrahydrolipstatin

(76) Inventors: Adelbert Bacher, Garching (DE); Peter Stohler, Lausen (CH); Wolfgang Weber, Grenzach-Wyhlen (DE)

Correspondence Address: HOFFMANN-LA ROCHE INC., PATENT LAW DEPARTMENT, 340 KINGSLAND STREET, NUTLEY, NJ, 07110

(21) Appl. No.: 10/124,115

(22) Filed: Apr. 17, 2002

Related U.S. Application Data
(63) Continuation of application No. 09/676,071, filed on Sep. 29, 2000.
Application No. 09/676,071 is a continuation of application No. 09/357,616, filed on Jul. 20, 1999, now abandoned.
Application No. 09/357,616 is a continuation of application No. 08/843,871, filed on Apr. 17, 1997, now abandoned.

(30) Foreign Application Priority Data
Apr. 26, 1996 (EP) 96106598.4

(51) Int. Cl. C12P 21/04

(52) U.S. Cl. 435/71.1

(57) ABSTRACT

A process for the fermentative production of lipstatin, which process comprises

a) aerobically cultivating a micro-organism of the order of actinomycetes which produces lipstatin, in an aqueous culture medium which is substantially free of fats and oils, and which contains suitable carbon and nitrogen sources and inorganic salts, until the initial growth phase is substantially finished, and

b) adding to the resulting broth, linoleic acid, optionally together with caprylic acid, [wherein part or the totality of the linoleic acid and/or of the caprylic acid can be replaced by the corresponding ester(s) and/or salt(s)], and N-formyl-L-leucine or preferably L-leucine, the linoleic acid or its ester(s) or salt(s) being stabilized by an antioxidant.

Lipstatin can be isolated from the broth and hydrogenated to tetrahydrolipstatin.

### CROSS REFERENCE TO RELATED APPLICATION

This is a continuation of copending patent Application Ser. No. 09/676,071, filed Sep. 29, 2000, which is a continuation of Ser. No. 09/357,616, filed Jul. 20, 1999 now Abandoned, which is a continuation of Ser. No. 08/843,871, filed Apr. 17, 1997, now Abandoned.

### BACKGROUND OF THE INVENTION

Field of the Invention

The invention relates to a novel fed-batch process for the fermentative production of lipstatin.

### SUMMARY OF THE INVENTION

The invention relates to a novel fed-batch process for the fermentative production of lipstatin, which process comprises

a) aerobically cultivating a micro-organism of the order of actinomycetes which produces lipstatin, in an aqueous culture medium which is substantially free of fats and oils, and which contains suitable carbon and nitrogen sources and inorganic salts, until the initial growth phase is substantially finished, and

b) adding to the resulting broth, linoleic acid, optionally together with caprylic acid, [wherein part or the totality of the linoleic acid and/or of the caprylic acid can be replaced by the corresponding ester(s) and/or salt(s)], and N-formyl-L-leucine or preferably L-leucine, the linoleic acid or its ester(s) or salt(s) being stabilized by an antioxidant.

### DETAILED DESCRIPTION OF THE INVENTION

The invention relates to a novel fed-batch process for the fermentative production of lipstatin, which comprises

a) aerobically cultivating a micro-organism of the order of actinomycetes which produces lipstatin, in an aqueous culture medium which is substantially free of fats and oils, and which contains carbon and nitrogen sources and inorganic salts, until the initial growth phase is substantially finished, and

b) adding to the resulting broth, linoleic acid and/or ester(s) or salt(s) thereof, with or without caprylic acid and/or ester(s) or salt(s) thereof, and N-formyl-L-leucine or L-leucine, wherein the linoleic acid or its ester(s) or salt(s) thereof is stabilized by an antioxidant.

Lipstatin, a fermentative process for its production, a process for its isolation from the broth and a process for its hydrogenation to tetrahydrolipstatin (also known as THL or orlistat, an anti-obesity agent) are described in U.S. Pat. No. 4,598,089.

The organism producing lipstatin as described in U.S. Pat. No. 4,598,089 is Streptomyces toxytricini Preobrazhenskaya & Sveshnikova (see Bergey's Manual of Determinative Bacteriology, 8^{th} edition, page 811). It was deposited on Jun. 14, 1983, at the Agricultural Research Culture Collection, Peoria, Ill. under the designation NRRL 15443. The process of the instant invention can be performed with this organism or with any other strain derived of it, such as a mutant strain selected for a better productivity. It can also be performed with other lipstatin producing organisms of the order actinomycetes, either belonging to the family of streptomycetes or belonging to another family within the order of actinomycetes.

When applying a fermentation system as described in U.S. Pat. No. 4,598,089, the fermentation broth after cultivation contains lipstatin in very small amounts of a few milligram per liter and it is difficult to isolate it by economically and technically feasible methods.

The present invention provides an improved process for the fermentative production of lipstatin, occurring in the fermentation broth with a higher concentration by using the fed-batch process described above. In the first step a) of this process the cells of the lipstatin producing micro-organism are grown in a basal medium. In the second step b) of this process, to this basal medium certain components are added, which either serve directly as biochemical precursors or undergo a short biochemical conversion and then serve as precursors of the biosynthetic pathway. By this system, the micro-organism is enabled to synthesize the desired product, lipstatin, in a much higher concentration.

The biosynthetic pathway leading to lipstatin is subject to a series of general control mechanisms, such as nitrogen repression. The addition of readily available nitrogen sources, especially amino acids and their derivatives, including L-leucine or N-Formyl-L-leucine, to the medium strongly represses the formation of lipstatin. In the present invention this is overcome by starting the addition of these amino acids only after the biosynthetic enzymes have been formed and thus are no longer repressed on a genetical level. In addition, L-leucine or N-formyl-L-leucine is added in such a way, that their concentration in the broth remains low.

Conveniently, the actinomycete producing lipstatin is grown in a suitable aqueous basal medium containing one or more carbon sources, such as starch, starch hydrolysates and sugars, for example, glucose and/or sucrose, glycerol, phospholipids, as well as one or more nitrogen sources, such as soybean flour, cotton seed flour, corn steep powder or corn steep liquor and yeast extract. Both carbon and nitrogen sources are supplied in such amounts that an abundant growth is enabled, typically in a range of 5 to 50 grams of each carbon source and of each nitrogen source per liter of medium. Further, macro- and micro-elements are added to the medium. Examples of macroelements are phosphate, magnesium and calcium ions. Examples of microelements (or traces) are iron, copper or cobalt ions. These macroelements and microelements might be contained in complex media, which in turn are contained in the carbon sources and particularly in the nitrogen sources in which the microorganisms are grown. Alternatively, the macroelements and microelements might be added as inorganic salts.

The aqueous culture medium is substantially free of fats and oils and contains less than 10 grams of triglycerides per liter of medium. The point at which the initial growth phase is finished is detectable by the exhaustion of the initial carbon source, e.g. glycerol, which can be measured by standard analytical techniques. It can also be seen by a sudden change of physiological parameters, such as a sharp decrease of both the oxygen uptake rate and of the carbon dioxide evolution rate. Practically, the addition of the biosynthetic precursors is started after the exhaustion of the initial carbon source. Conveniently, linoleic acid and/or caprylic acid and/or their esters or salts are fed at such a rate as to be freely available in the broth but so that their accumulation is prevented, particularly at a rate of 10 to 1000, preferably of 100 to 300 mg per liter and hour. The feeding of linoleic acid and/or caprylic acid and/or their salts or of their esters is preferably conducted so that their concentration in the broth remains inferior to 1000, preferably inferior to 300 mg per liter. Preferably, the feeding of linoleic acid and/or caprylic acid and/or their salts or esters is discontinued before starting the isolation of lipstatin. Conveniently, the linoleic acid and/or its ester(s) or salt(s) and caprylic acid and/or its ester(s) or salt(s) are added to the broth in a ratio of 1 to 10, preferably 1.5 to 3 parts per weight of linoleic acid and/or esters or salts being added for 1 part of caprylic acid and/or its esters or salts. Conveniently, N-formyl-L-leucine or preferably L-leucine is added to the broth at a rate of 1 to 100, preferably 5 to 50 mg per liter of broth per hour, so that its concentration remains less than 25 millimolar.

Examples of salts and esters which can be substituted for a part (or for the totality) of the linoleic acid or of its mixture with caprylic acid are alkaline or alkaline earth metal salts, for example, sodium, potassium, calcium or magnesium salts, and lower alkyl esters, for example, methyl esters, or glycerides.

In order to prevent the oxidation of linoleic acid (or of its ester(s) or salt(s)) it is mixed with an antioxidant, such as ascorbyl palmitate, tocopherol, lecithin, or mixtures thereof, and/or a radical trapping agent, such as BHA (tert.-butyl-4-hydroxy-anisol) or BHT (2,6-ditert.-butyl-p-cresol).

The invention further relates to a process for the production of tetrahydrolipstatin, which process comprises

a) aerobically cultivating a micro-organism of the order of actinomycetes which produces lipstatin, in an aqueous culture medium which is substantially free of fats and oils, and which contains suitable carbon and nitrogen sources and inorganic salts, until the initial growth phase is substantially finished, and

b) adding to the resulting broth, linoleic acid, preferably together with caprylic acid, [wherein part or the totality of the linoleic acid and/or of the caprylic acid can be replaced by the corresponding ester(s) and/or salt(s)], and N-formyl-L-leucine or preferably L-leucine, the linoleic acid or its ester(s) or salt(s) being stabilized by an antioxidant,

c) isolating lipstatin from the broth and hydrogenating lipstatin to tetrahydrolipstatin.

The isolation of the lipstatin from the fermentation broth can be carried out according to methods which are known per se and which are familiar to any person skilled in the art. For example, it can be carried out as follows:

After completion of the fermentation, the fermentation broth is centrifuged. The resulting cell mass can then be treated with a lower alkanol such as methanol or ethanol, and extracted with the same solvent. The centrifugate can be extracted with a suitable organic solvent (for example, with methylene chloride or ethyl acetate). The material produced from the extracts contains the desired lipstatin and can be enriched and purified by chromatographic methods, for example, as described in U.S. Pat. No. 4,598,089.

The hydrogenation of lipstatin to tetrahydrolipstatin can be carried out according to methods which are known per se, for example, as described in U.S. Pat. No. 4,598,089, in the presence of a suitable catalyst. Examples of catalysts which can be used are palladium/carbon, platinum oxide, palladium and the like. Suitable solvents are, for example, lower alcohols such as methanol and ethanol. The hydrogenation is preferably carried out at low hydrogen pressures and at room temperatures.

### EXAMPLE 1

a) A seed culture is prepared consisting of the following pre-culture medium: 10 g of defatted soy flour, 10 g of glycerol, 5 g of yeast extract and water to make 1 l. The pH is adjusted to 7.0 with NaOH 28%, giving a pH of 6. 8 after sterilization. 100 ml of this medium is filled into a 500 ml Erlenmeyer flask, closed with a cotton plug and sterilized. It is then inoculated with a loopful of spores of Streptomyces toxytricini strain NRRL 15443 and subsequently incubated under shaking at 27° C. for 24 hours.

b) 100 ml of this seed culture is used to inoculate a fermentor with a vessel size of 14 l containing 8 l of a production medium containing per liter: 32 g of defatted soybean flour, 20 g of glycerol, 14 g of lecithin, 0.25 ml of polypropylene glycol as an antifoam agent, whereas the pH is adjusted to 7.4 with NaOH 28%. The medium contains less than 5 grams per liter of triglycerides.

c) After a growth phase of 47 hours, the feeding is started. It consists of the fatty acids linoleic acid and caprylic acid, whereby linoleic acid is stabilized by the addition of 0.2% (w/w) of an antioxidant (RONOXAN A) consisting of 70% (w/w) of lecithin, 25% of ascorbyl palmitate and 5% of tocopherol. These fatty acids are added at a rate of 136 to 190 mg per liter and hour, and the rate of addition is adjusted in such a way that the concentration of each linoleic acid and caprylic acid remains below 70 mg per liter. Totally added in the course of the fermentation are 108 grams of linoleic acid and 54 g of caprylic acid. The L-leucine is added at a rate of 14.4 mg per liter and hour, as an aqueous solution containing 80 g of L-leucine per 1 of feed, the pH being adjusted to 11 with NaOH 28%. A total of 10.2 grams of L-leucine is added.

The culture medium after seeding with the aforementioned seed culture and while feeding with the aforementioned fatty acids and L-leucine is incubated under stirring, and aeration at a rate of 4 l of air per minute to keep the culture aerobic. The pH is maintained in a range of 6.1 to 7.3 by the automated addition of sulfuric acid or sodium hydroxide solution. The dissolved oxygen concentration is prevented to be less than 10% of the saturation concentration by adjusting the stirrer speed. At harvest time, the culture is practically free of linoleic acid and caprylic acid. The titer of lipstatin, that is, its concentration in the culture medium, is 150 mg/l after an incubation period of 138 hours.

### EXAMPLE 2

The same seed culture as in Example 1a) above is used to inoculate a 14 l fermentor with a production medium as described in U.S. Pat. No. 4,598,089, namely the production medium N 7 in Example 1 thereof. This medium contains, per 8 liters: 80 g of potato starch, 40 g of glucose, 80 g of ribose, 40 g of glycerol, 16 of peptone, 160 g of soybean flour, 16 g of ammonium sulfate. An antifoam agent (0.25 ml of polypropylene glycol per liter of medium) is added as in example 1b) above. The pH is adjusted to 7.0 with NaOH 28% before sterilization. Incubation is carried out aerobically, while stirring at 400 rpm and with an aeration rate of 4 l of air per minute.

After incubation, a concentration of lipstatin of less than 10 mg/l was found in the culture medium.

### EXAMPLE 3

a) A seed culture, prepared as in Example 1, is inoculated with spores of a mutant strain designated strain R 4569 which was derived from Streptomyces toxytricini strain NRRL 15443 and which was selected for an increased production of lipstatin, and subsequently incubated under shaking at 27° C. for 24 hours.

b) 100 ml of this seed culture is used to inoculate a fermentor with a vessel size of 14 l containing 8 l of a medium containing per liter: 48 g of defatted soybean flour, 30 g of glycerol, 100 mg of CaCO₃ and 1 ml of a polypropyleneglycol antifoam agent. The medium contains less than 5 grams per liter of triglycerides.

c) After a growth phase of 34 hours, the feeding is started. It consists of linoleic acid, which is stabilized by the addition of 0.3% (w/w) of RONOXAN A. Linoleic acid is added at a rate of 140 to 210 mg per liter and hour, and the rate of addition is adjusted in such a way that its concentration remains below 1000 mg per liter. Totally added in the course of the fermentation are 256 grams of linoleic acid. The L-leucine is added at a rate of 14-21 mg per liter and hour, as an aqueous solution containing 80 g of L-leucine per liter of feed, the pH being adjusted to 11 with NaOH 28%. A total of 25.36 grams of L-leucine is added.

The culture medium after seeding with the aforementioned seed culture and while feeding with the aforementioned fatty acid and L-leucine, is incubated under stirring and aeration to keep the culture aerobic, whereby the pH is maintained in a range of 6.8 to 7.0 by the automated addition of sulfuric acid or sodium hydroxide solution. The dissolved oxygen concentration is prevented to be less than 30% saturation by adjusting the stirrer speed. The titer of lipstatin, that is, its concentration in the culture medium, produced under these conditions is 9106 mg/l after an incubation period of 184 hours.

1. A process for the fermentative production of lipstatin, which process comprises
a) aerobically cultivating a micro-organism of the order of actinomycetes which produces lipstatin, in an aqueous culture medium which is substantially free of fats and oils, and which contains carbon and nitrogen sources and inorganic salts, until the initial growth phase is substantially finished, and
b) adding linoleic acid and/or ester(s) or salt(s) thereof, to the resulting broth, with or without caprylic acid and/or ester(s) or salt(s) thereof, and N-formyl-L-leucine or L-leucine, wherein the linoleic acid and/or ester(s) or salt(s) thereof, is stabilized by an antioxidant.

2. A process according to claim 1, wherein the micro-organism of the order of actinomycetes is of the family of streptomycetes, and the aqueous culture medium contains less than 10 grams of triglycerides per liter of medium.

3. A process according to claim 2, wherein the linoleic acid and/or ester(s) or salt(s) thereof, with or without the caprylic acid and/or ester(s) or salt(s) thereof, is added to the broth at such a rate as to be freely available in the broth but so that their accumulation is prevented.

4. A process according to claim 3, wherein the linoleic acid and/or ester(s) or salt(s) thereof, with or without the caprylic acid and/or ester(s) or salt(s) thereof, is added to the broth at a rate of 10 to 1000 mg per liter and hour.

5. A process according to claim 4, wherein linoleic acid and/or ester(s) or salt(s) thereof, with or without the caprylic acid and/or ester(s) or salt(s) thereof, is added to the broth at a rate of 100 to 300 mg per liter and hour.

6. A process according to claim 3, wherein the addition of linoleic acid with or without caprylic acid and/or their salts or esters is conducted so that the concentration of linoleic acid with or without caprylic acid and/or their salt(s) or ester(s) in the broth remains less than 1000 mg per liter, and the addition is discontinued before lipstatin is isolated.

7. A process according to claim 6, wherein the addition of linoleic acid with or without caprylic acid and/or their salt(s) or ester(s) is conducted so that the concentration of linoleic acid with or without caprylic acid and/or their salt(s) or ester(s) in the broth remains less than 300 mg per liter, and the addition is discontinued before lipstatin is isolated.

8. A process according to claim 6, wherein the linoleic acid and caprylic acid and/or ester(s) or salt(s) thereof are added to the broth in a ratio of 1 to 10 parts per weight of linoleic acid and/or ester(s) or salt(s) thereof to 1 part per weight of caprylic acid and/or ester(s) or salt(s) thereof.

9. A process according to claim 8, wherein the linoleic acid and caprylic acid and/or their esters or salts are added to the broth in a ratio of 1.5 to 3 parts per weight of linoleic acid and/or ester(s) or salt(s) thereof to 1 part per weight of caprylic acid and/or ester(s) or salt(s) thereof.

10. A process according to claim 8, wherein the N-formyl-L-leucine or L-leucine is added to the broth at a rate of 1 to 100 mg per liter of broth per hour, so that its concentration remains less than 25 millimolar.

11. A process according to claim 10, wherein the N-formyl-L-leucine or L-leucine is added to the broth at a rate of 5 to 50 mg per liter of broth per hour, so that its concentration remains less than 25 millimolar.

12. A process for the production of tetrahydrolipstatin, which process comprises
a) aerobically cultivating a micro-organism of the order of actinomycetes which produces lipstatin, in an aqueous culture medium which is substantially free of fats and oils, and which contains carbon and nitrogen sources and inorganic salts, until the initial growth phase is substantially finished, and
b) adding linoleic acid and/or ester(s) or salt(s) thereof, to the resulting broth, with or without caprylic acid and/or ester(s) or salt(s) thereof, and N-formyl-L-leucine or L-leucine, wherein the linoleic acid and/or its ester(s) or salt(s) thereof is stabilized by an antioxidant to form lipstatin and,
c) isolating the lipstatin from the broth and hydrogenating lipstatin to tetrahydrolipstatin.
